(19) United States
(12) Plant Patent Application Publication
(10) Pub. No.: US 2017/0231133 P1
(43) Pub. Date: Aug. 10, 2017

(54) Aronia plant named 'UCONNAM166'

(50) ;

(71) Applicant: University of Connecticut, Farmington, CT (US)

(72) Inventors: Mark Henry Brand, Willington, CT (US); Bryan Allan Connolly, Mansfield Center, CT (US)

(73) Assignee: University of Connecticut, Farmington, CT (US)

(21) Appl. No.: 14/998,768

(22) Filed: Feb. 10, 2016

(51) Int. Cl. A01H 5/02

(52) U.S. Cl. PLT/156

(57) ABSTRACT

A new and distinct Aronia cultivar named ‘UCONNAM166’ is disclosed, characterized by a distinctly upright plant form, reaching a mature height between 4 to 5 feet. Vigorous but controlled plant growth. Multicolored Fall foliage. Plants are adaptable to a variety of landscape uses, including hedging or mass planting. Triploid plants produce very few, or no fruits. The new variety is a Aronia, normally produced as an outdoor landscape plant.

Latin name of the genus and species: Aronia melanocarpa.

Variety denomination: ‘UCONNAM166’.

### BACKGROUND OF THE INVENTION

The new Aronia cultivar is a product of a planned breeding program conducted by the inventors, Mark Henry Brand and Bryan Allan Connolly in Storrs, Conn. The objective of the breeding program was to produce new Aronia varieties like ‘Viking’ but with narrower and shorter form. The cross resulting in this new variety was made during May of 2010.

The seed parent is the, unpatented variety referred to as Aronia melanocarpa ‘UC012’. The pollen parent is the unpatented variety Aronia mitschurinii ‘Viking’. The new variety was discovered in June of 2011 by the inventors in a group of seedlings resulting from the 2010 crossing, in a greenhouse in Storrs, Conn.

Asexual reproduction of the new cultivar ‘UCONNAM166’ first by tissue culture was first performed during the June of 2011; at a laboratory in Storrs, Conn. Subsequent propagation by softwood cuttings has shown that the unique features of this cultivar are stable and reproduced true to type in 3 successive generations.

### SUMMARY OF THE INVENTION

The cultivar ‘UCONNAM166’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘UCONNAM166’ These characteristics in combination distinguish ‘UCONNAM166’ as a new and distinct Aronia cultivar:
1. Distinctly upright form, 4-5′ tall by 3′ wide. Shoots are vertical.
2. Leaves are larger than typical for Aronia meloancarpa. Leaves are slightly smaller than leaves of Aronia mitschurinii ‘Viking’, but have a similar appearance to ‘Viking’.
3. Vigorous growth, but not as vigorous as Aronia mitschurinii ‘Viking’.
4. Fall foliage color is multi-colored, orange, red, and yellow.
5. Adaptable, easy to grow, full sun to partial shade.
6. Good for hedge applications or mass planting in challenging landscape situations.
7. White flowers in clusters in early may (Connecticut).
8 Triploid plants with little or no fruit production.

### PARENT COMPARISON

Plants of the new cultivar ‘UCONNAM166’ are similar to plants of the seed parent, Aronia melanocarpa ‘UC012’in most horticultural characteristics, however, plants of the new cultivar ‘UCONNAM166’ differ in the following;
1. 4-5′ tall by 3′ wide, while the seed parent is 1-2′ tall and 3-4′ wide.
2. Upright, vertical habit, no rhizomes, while the seed parent is prostrate, with a highly rhizomatous growth habit.
3. Light to moderate flowering, while the seed parent is moderate to heavy flowering.
4. Leaves are large sized, while the seed parent has small to medium sized leaves.
5. Minimal or no berries, while the seed parent has black berries.

Plants of the new cultivar ‘UCONNAM166’ are similar to plants of the pollen parent, Aronia melanocarpa ‘UC012’in most horticultural characteristics, however, plants of the new cultivar ‘UCONNAM166’ differ in the following;
1. 4-5′ tall by 3′ wide, while the pollen parent is 6-8′ tall and 6-8′ wide.
2. Upright, vertical habit, while the pollen parent is of somewhat loose, open spreading form.
3. Short internodes, while the pollen parent has long internodes.
4. Leaves are large sized, while the pollen parent has very large leaves.
5. Minimal or no berries, while the pollen parent has black berries.

### COMMERCIAL COMPARISON

Plants of the new cultivar ‘UCONNAM166’ can be compared to the unpatented commercial variety Aronia ‘Morton’ (Iroquois Beauty). These varieties are similar in most horticultural characteristics; however, ‘UCONNAM166’ differs in the following:
1. 4-5′ tall by 3′ wide, while the comparator is 5-6′ tall and 5-7′ wide.
2. Upright, vertical habit, no rhizomes, while the comparator is upright spreading habit with rhizomes.
3. Leaves are large sized, while the comparator has medium sized leaves.
4. Minimal or no berries, while the comparator has black berries.

Plants of the new cultivar ‘UCONNAM166’ can also be compared to the unpatented commercial variety Aronia ‘Autumn Magic’. These varieties are similar in most horticultural characteristics; however, ‘UCONNAM166’ differs in the following:
1. 4-5′ tall by 3′ wide, while the comparator is 5-6′ tall and 5-7′ wide.
2. Upright, vertical habit, no rhizomes, while the comparator is upright spreading habit with rhizomes.
3. Leaves are large sized, while the comparator has medium sized leaves.
4. Minimal or no berries, while the comparator has black berries.

### BRIEF DESCRIPTION OF THE PHOTOGRAPHS

The accompanying photograph in FIG. 1 illustrates in full color typical plants of ‘UCONNAM166’ grown outdoors, in Mansfield, Conn.

FIG. 2 illustrates in full color a typical inflorescence of ‘UCONNAM166’. Age of the plant photographed is approximately 3 years. The photographs were taken using conventional techniques and although colors may appear different from actual colors due to light reflectance it is as accurate as possible by conventional photographic techniques.

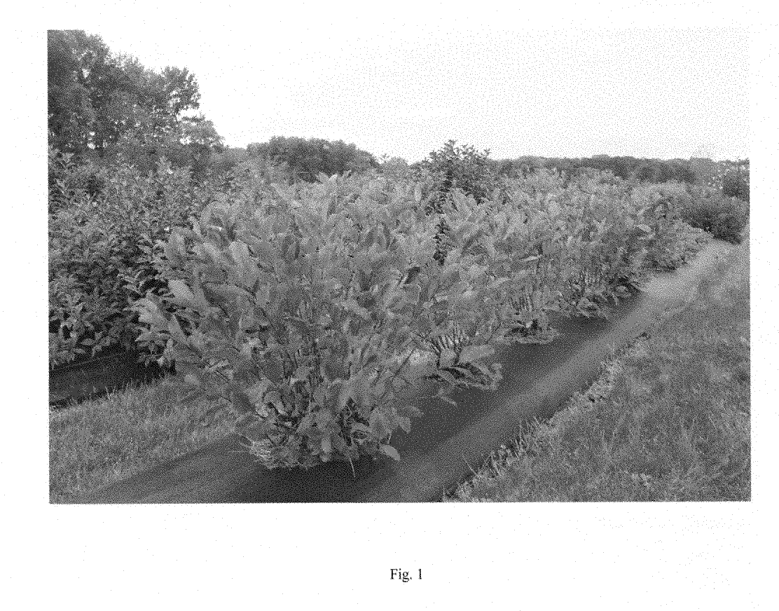

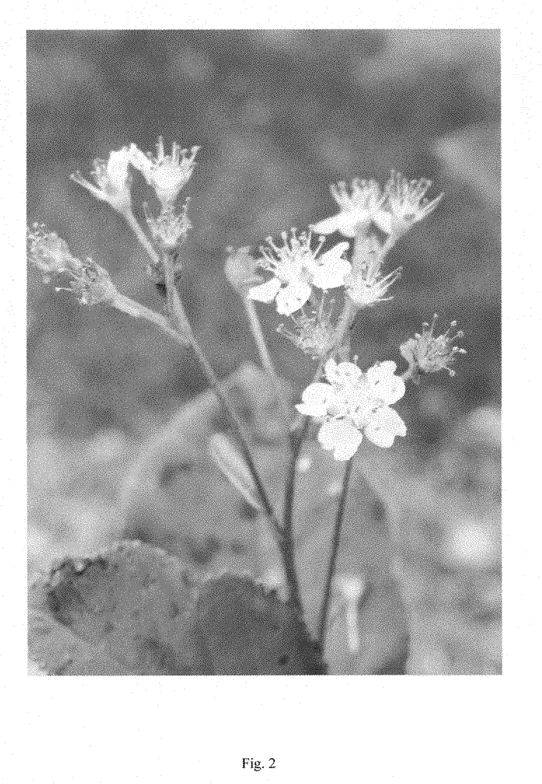

### DETAILED BOTANICAL DESCRIPTION

In the following description, color references are made to The Royal Horticultural Society Colour Chart 1996 except where general terms of ordinary dictionary significance are used. The following observations and measurements describe ‘UCONNAM166’ plants grown in poly house in Grand Haven, Mich., under natural lighting. Measurements were taken during April of 2015. The plants were 2-3 years old in 3 gallon containers. The growing temperature ranged from 5° C. to 27° C. Measurements and numerical values represent averages of typical plant types.
Botanical classification: Aronia melanocarpa ‘UCONNAM166’.
Propagation:
   Time to initiate roots.—About 3 weeks at approximately 24° C.
   Time to produce a rooted young plant.—About 8 weeks at 24° C.
   Roots.—Moderate density moderate branching. Medium thickness, fibrous and white in color, not accurately measured with the R.H.S. Chart.
Plant:
   Plant type.—Perennial shrub.
   Age of plant described.—Approximately 2-3 years old.
   Growth habit.—Upright and compact.
   Pot size of plant described.—3 gallon pot.
   Appropriate containers sizes.—1, 2, and 3 gallon pots.
   Overall plant shape.—Upright.
   Height.—92 cm.
   Plant spread.—92 cm.
   Growth rate.—Moderate.
   Plant vigor.—Good.
   Branching characteristics.—Upright occurring branches, with basal branching.
   Length of primary lateral branches.—90 cm.
   Diameter of lateral branches.—5 mm.
   Quantity of lateral branches.—20-30.
   Internode length.—4.5 cm.
   Stem.—Stem Appearance: Round Stem Strength: Moderate Stem Color: New growth: Near RHS Greyed-Orange 166B 1-year-old growth: Near RHS Greyed-Orange 177B Old Growth: Near RHS Yellow-Green 152A Pubescence: Yes Aspect: 80-90° very upright.
   Other plant characteristics.—Lenticels: 1-2 mm Near RHS Greyed-Orange 164B.
Foliage:
   Leaf.—Arrangement: Alternate, single Leaf shape: Elliptic Average Length: 9-10 cm Average Width: 5-6 cm Apex: Acute to cuspidate Base: Between cuneate and obtuse. Margin: Crenate Texture of top surface: Rugose/bullate (smooth/waxy) Texture of bottom surface: Slightly rough/waxy Leaf side with pubescence: Under side Color: Young foliage upper side: Near RHS Yellow-Green 144A Young foliage under side: Near RHS Yellow-Green 146D Mature foliage upper side: Near RHS Green 137A Mature foliage under side: Near RHS Yellow-Green 146C Venation: Pattern: Pinnate Color upper side: Midrib: Near RHS Yellow-Green 146D Veins: Near RHS Green 137A Color under side: Midrib: Near RHS Yellow-Green 146D Veins: Near RHS Yellow-Green 146C Petiole: Average Length: About 1 cm Diameter: 1-3 mm Petiole color upper side: Near RHS Yellow-green 146D Petiole color lower side: Near RHS Yellow-green 146D Petiole Texture upper side: Canaliculate/smooth Petiole Texture lower side: Canaliculate/smooth.
Flower:
   Natural flowering season.—Spring.
   Flower arrangement.—Corymb.
   Inflorescence type and habit.—Rotate, unguiculate.
   Inflorescence height.—2-3 cm.
   Inflorescence width.—2-3 cm.
   Quantity of flowers per inflorescence.—5-10.
   Quantity of flowers per lateral stem.—30-40.
   Quantity of flowers per plant.—80-300.
   Flower longevity on plant.—1-2 weeks.
   Individual flowers.—Flower form: Rotate. Flower shape: Radial Flower aspect: Upright Flowering habit: Unguiculate.
   Size.—Diameter: 12 mm Length: 12 mm Depth: 6 mm.
   Flower other characteristics.—Persistence: Self cleaning. Fragrance: Slight.
   Petal.—Petal arrangement: Whorled Petal appearance: Unguiculate Number of petals per flower: 5 Petal fused: No Petal Shape: Circular Petal Base: Cuspidate Margin: Entire Tip shape: Rounded Length: 5 min Width: 4 mm Texture: Upper: Glabrous Lower: Glabrous.
   Petal color.—When opening: Upper surface: Near RHS White 155B Lower surface: Near RHS White 155B with some Red-Purple 62C Fully opened: Upper surface: Near RHS White 155B Lower surface: Near RHS White 155B.
   Bud.—Shape: Spherical Length: 5 mm Diameter: 4 mm Color: Near RHS White 155B and Greyed-Purple 186B.
   Sepals.—Arrangement: Radial Number: 5 Shape: Deltate Base: Truncate Margin: Entire Tip shape: Acute Length: 1 mm Width: 1 mm Color upper: Near RHS Green 139D Color lower: Near RHS Green 139C Texture: Upper: Pubescent Lower: Glabrous.
   Calyx.—Shape: Radial Length: 3 mm Diameter: 5.5 mm.
   Peduncle.—Length: 7 mm Diameter: 1 mm Color: Near RHS Yellow-Green 146D Strength: Good Texture: Glabrous.
Reproductive organs:
   Stamens.—Number: 18 Filament length: 4 mm Filament color: Near RHS White 155B.
   Anthers.—Shape: Dorsifixed Length: 1 mm Color: Near RHS Red-Purple 59B Pollen Color: Brown Pollen Amount: Little-moderate.
   Pistil.—Number: 4 Length: 3 mm Style: Length: 1 mm Color: Near RHS Yellow-Green 146D Stigma: Shape: Syncarpous fused stigma with 3-5 lobes Color: Near RHS Yellow-Green 146D Ovary Color: Near RHS Yellow-Green 146D.
Other characteristics:
   Disease/pest resistance.—Typical of the species but better mildew resistance.
   Temperature tolerance.—The new variety tested to temperatures between −25° C. to 40 ° C.

What is claimed is:

1. A new and distinct cultivar of Aronia plant named ‘UCONNAM166’ as herein illustrated and described.
